(19) United States
(12) Patent Application Publication
(10) Pub. No.: US 2024/0224950 A9
(43) Pub. Date: Jul. 11, 2024

(54) INCUBATION TRAY FOR BEES

(71) Applicants: David TORRIE, Grassy Lake (CA); Michael K. MADAN, Reno, NV (US)

(72) Inventors: David TORRIE, Grassy Lake (CA); Michael K. MADAN, Reno, NV (US)

(21) Appl. No.: 18/142,029

(22) Filed: May 2, 2023

(30) Foreign Application Priority Data
May 2, 2022 (CA) 3157857

(51) Int. Cl. A01K 49/00

(52) U.S. Cl. CPC A01K 49/00

(57) ABSTRACT

A set of trays for incubation includes a cover tray and a screened tray. The cover tray has a closed end surrounded by a peripheral sidewall that defines a cavity. The screened tray includes a screened panel surrounded by a containment flange that surrounds and extends outward from a first face of the screened panel, and a support structure that extends outward from a second face of the screened panel opposite the containment flange. The support structure has apertures that permit airflow through the support structure. The containment flange and support structure are sized to engage overlying and underlying trays in alignment and at an angle.

### TECHNICAL FIELD

This relates to an incubation tray, and in particular, an incubation tray and lid for incubating leafcutter bees.

### BACKGROUND

Leafcutter bees are often used in agriculture to promote pollination of crops. This often involves managing the bees through their lifecycle, including incubation from larva to adult. This is often done in trays with screened panels. U.S. Pat. No. 9,801,358 (Allan) entitled “Method and apparatus for field incubation of bees” is an example of screened trays used to help incubate bees.

### SUMMARY

According to an aspect, there is provided an set of trays, comprising a cover tray having a closed end surrounded by a peripheral sidewall that defines a cavity, the cavity having an opening opposite the closed end, and a a screened tray. The screened tray comprises a screened panel that permits airflow therethrough, and a containment flange that surrounds and extends outward from a first face of the screened panel. The containment flange is sized to engage the opening of the cover tray such that the screened panel covers and permits airflow through the opening of the cavity. A support structure extends outward from a second face of the screened panel opposite the containment flange. The support structure has apertures that permit airflow through the support structure, the support structure being sized to engage the closed end of the cover tray at a position spaced from the screened panel such that the apertures are disposed between the closed end of the cover tray and the screened panel.

According to other aspects, the set of trays may include one or more of the following features, alone or in combination: the containment flange may be sized to nest within the opening of the cavity and immediately adjacent to an inner surface of the peripheral sidewall; the support structure may surround the screened panel and comprises a remote edge; the closed end of the cover tray may be sized to nest within the support structure and comprises a peripheral shoulder that is sized to engage the remote edge of the support structure; each of the cover tray and the screened tray may be rectangular; the closed end may further comprise indents and the remote edge of the support structure comprises stops, the indents and stops cooperating to support the cover tray perpendicularly relative to the screened tray; the containment flange of the screened tray may comprise indents that cooperate with the stops of the support structure to support a plurality of screened trays stacked transversely; the screened tray may comprise a stepped profile that engages the peripheral sidewall of the cover tray and supports the screened panel of the screened tray at the opening of the cavity of the cover tray; and the screened tray may be invertible, such that the containment flange and the screened panel define a containment tray cavity.

According to an aspect, there is provided a stack of trays, comprising at least an underlying set of trays and an overlying set of trays, each of the underlying set of trays and the overlying set of trays comprising a cover tray and a screened tray, wherein: each cover tray comprises a length, a width, and a closed end surrounded by a peripheral sidewall that defines a cavity having an opening opposite the closed end; and each screened tray comprises a length and a width substantially the same as the length and the width of the cover tray. The screened tray further comprises a screened panel that permits airflow therethrough; a containment flange that surrounds and extends outward from a first face of the screened panel, the containment flange being received within the opening of the cover tray such that the screened panel covers and permits airflow through the opening of the cavity; and a support structure that extends outward from a second face of the screened panel opposite the containment flange, the support structure having apertures that permit airflow through the support structure, the support structure being configured to engage the closed end of the cover tray in a first position in which the cover tray is substantially aligned with the screened tray and the cover tray is spaced from the screened panel such that the apertures are disposed between the closed end of the tray and the screened panel, and in a second position in which the cover tray is substantially perpendicular to the screened tray. The closed end of the cover tray of the overlying set of trays engages the support structure of the screened tray of the underlying set of trays.

According to other aspects, the stack of trays may further comprise one or more of the following aspects, alone or in combination: of claim 8, wherein the containment flange of each screened tray is sized to nest within the opening of the cavity and immediately adjacent to an inner surface of the peripheral sidewall of each cover tray; the support structure may surround the screened panel, and the closed end of each cover tray may be configured to nest within the support structure with a peripheral shoulder that is sized to engages a remote edge of the support structure; the closed end of each cover tray may further comprise indents and the remote edge of the support structure of each screened tray comprises stops, the indents and stops cooperating to support the cover tray of the overlying set of trays in the second position on the screened tray of the underlying set of trays; each screened tray may comprise a stepped profile that is sized to engage the peripheral sidewall of each cover tray and support the screened panel of each screened tray at the opening of the cavity of each cover tray; each screened tray may be invertible, such that the containment flange and the screened panel define a containment tray cavity in a first orientation and encloses the cavity of the cover tray in a second orientation.

According to an aspect, there is provided a method of incubating bees, comprising: placing bee larva in a plurality of screened trays, wherein each screened tray may be as described above; stacking the plurality of screened trays in a first stack such that adjacent trays in the stack are positioned in a transverse orientation; and at a predetermined stage of development of the plurality of bee larva, forming a plurality of sets of trays by installing a cover tray on each of the screened trays, each cover tray comprising a length and a width that are substantially the same as the length and the width of the screened tray, and a closed end surrounded by a peripheral sidewall that defines a cavity having an opening opposite the closed end, wherein the containment flange of the screened tray is received within the cavity of the cover tray.

According to other aspects, the plurality of sets of trays may be stacked in a second stack in which the support structure and the closed ends of adjacent sets of trays are engaged and such that adjacent sets of trays are positioned in a transverse orientation, wherein the closed end of each cover tray has a third edge profile that cooperates with the second edge profile of the support structure in the transverse orientation; at a further stage of development: placing the plurality of sets of trays in a third stack in which the support structures and the closed ends of adjacent sets of trays are engaged such that adjacent sets of trays are aligned; transporting the third stack to an area to be pollinated; and for each set of trays and with the cover tray positioned below the screened tray such that developed bee larva are in the cover tray, removing the screened tray and placing the cover tray at a desired location in the area to be pollinated; when the plurality of sets of trays are placed in the third stack, the closed ends of the cover trays may nest within the support structures of an adjacent set of trays, the closed ends may comprise a peripheral shoulder that engages a remote end of the support structure; the containment flange of each screened tray may be sized to nest within the opening of the cavity of the cover tray and immediately adjacent to an inner surface of the peripheral sidewall; and in the first stack, the containment flange and the screened panel may define a containment tray cavity of each screened tray.

In other aspects, the features described above may be combined together in any reasonable combination as will be recognized by those skilled in the art.

### BRIEF DESCRIPTION OF THE DRAWINGS

These and other features will become more apparent from the following description in which reference is made to the appended drawings, the drawings are for the purpose of illustration only and are not intended to be in any way limiting, wherein:

FIG. 1 is a perspective, exploded view of a set of trays.

FIG. 2 is a perspective view of a screened tray.

FIG. 3 is a top plan view of the screened tray of FIG. 2.

FIG. 4 is a side elevation view in cross section of the screened tray of FIG. 2.

FIG. 5 is a side elevation view of trays stacked perpendicularly.

FIG. 6 is a side elevation view of a stack of trays stacked in alignment.

FIG. 7 is a side elevation view of screened trays stacked perpendicularly in a containment orientation.

FIG. 8 is top plan view of the screened trays of FIG. 7.

### DETAILED DESCRIPTION OF PREFERRED EMBODIMENTS

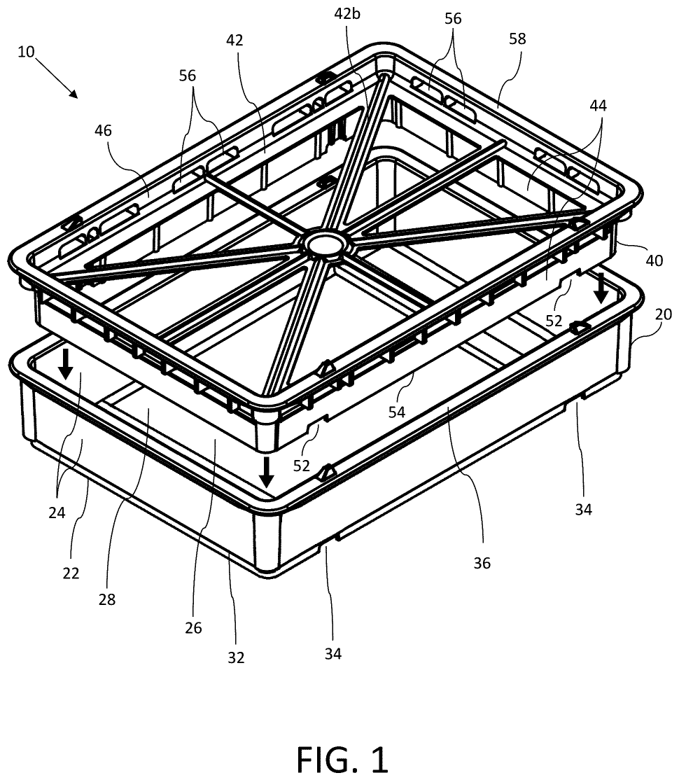

A set of trays, generally identified by reference numeral 10, will now be described with reference to FIG. 1 through 8. Set of trays 10 may be used for the incubation of leafcutter bees or other solitary bees or insects that have a similar development cycle.

Referring to FIG. 1, set of trays 10 includes a cover tray 20 and a screened tray 40. Cover tray 20 has a closed end 22 surrounded by a peripheral sidewall 24 that defines a cavity 26. Cavity 26 has an opening 28 opposite closed end 22. Opening 28 may be defined by an edge 36 of peripheral sidewall 24. Closed end 22 may have indents 34 on an outer surface of cover tray 20 that are useful for stabilizing cover trays 20 and screened tray 40 during stacking. Indents 34 may have a width that corresponds to the thickness of a remote edge 58 of screened tray 40 to help with alignment and reduce movement when stacked.

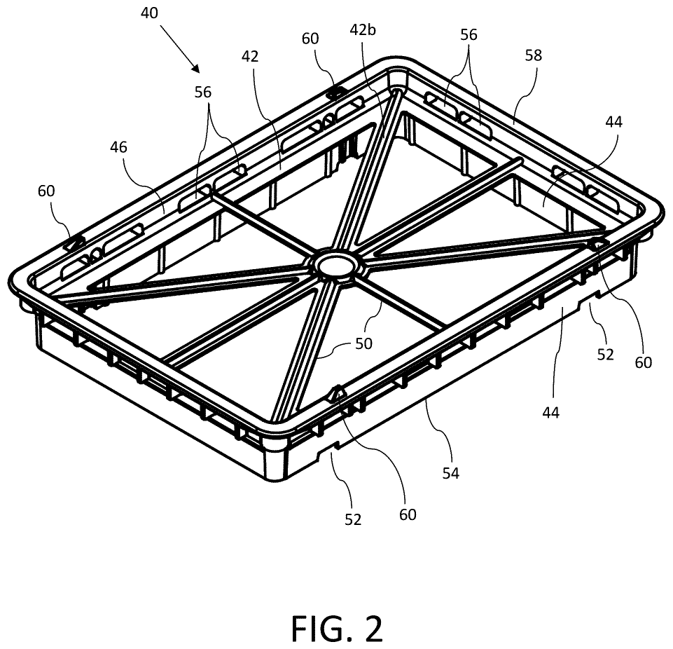

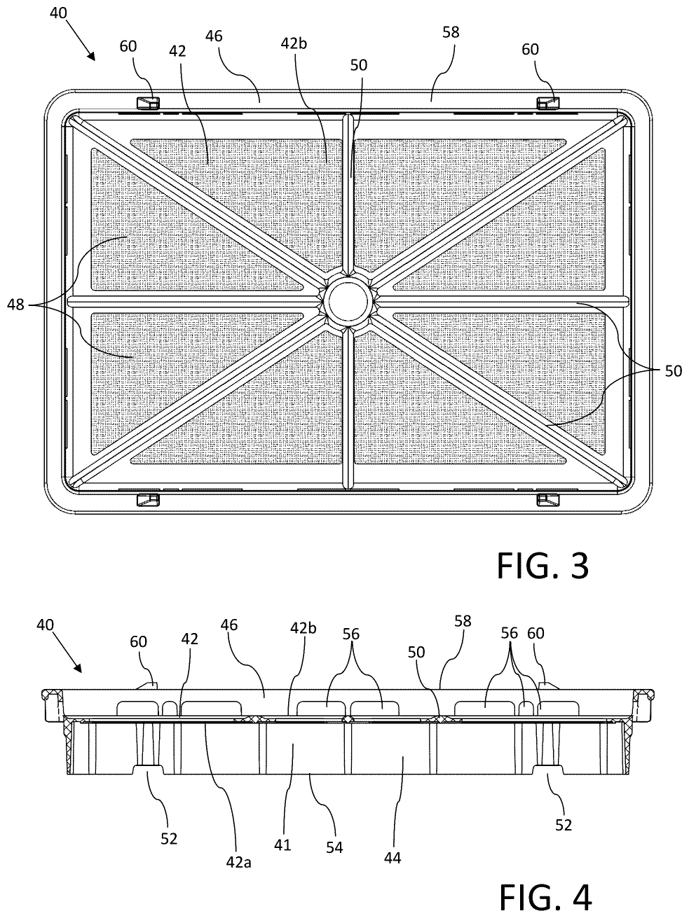

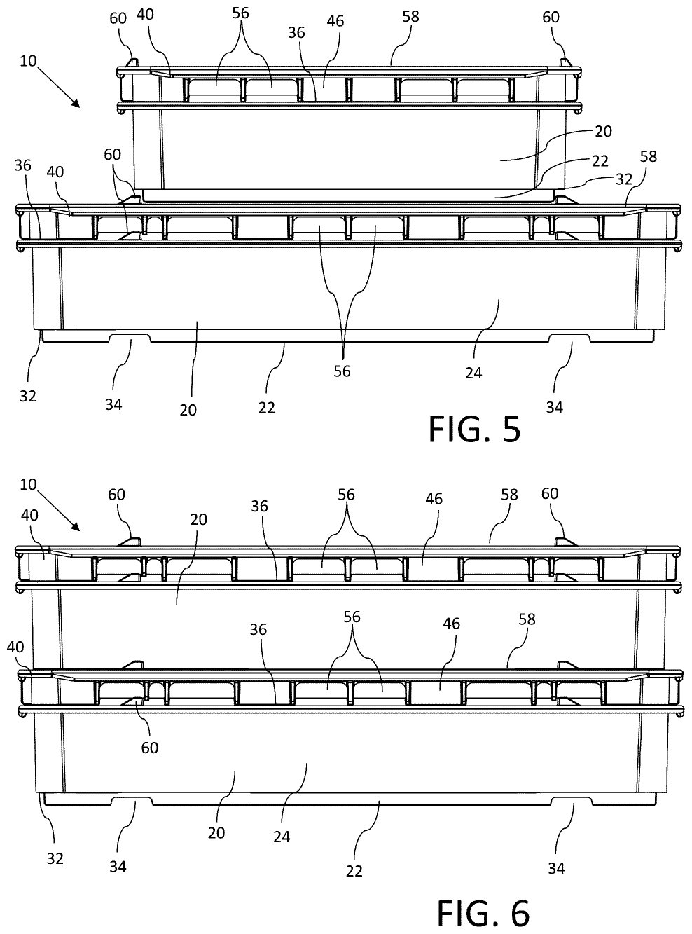

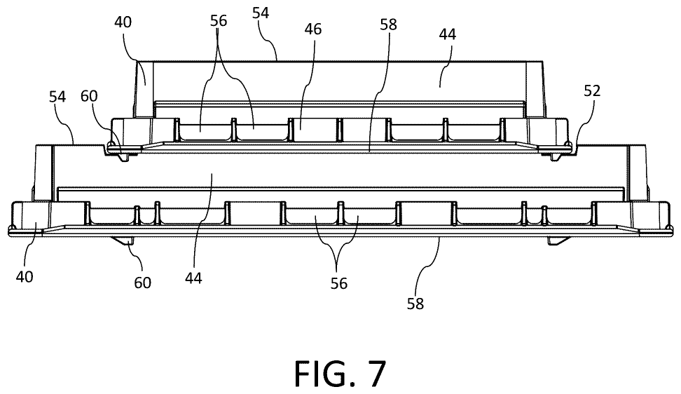

Referring to FIGS. 2 and 3, screened tray 40 has a screened panel 42 that permits airflow therethrough, a containment flange 44 formed as a depending skirt, and a support structure 46 that extends out from screened panel 42 opposite containment flange 44. Referring to FIG. 3, screened panel 42 may have a plurality of screen segments 48 attached between a plurality of screen supports 50. In FIGS. 1 and 2, screened panel 42 is shown without screen segments 48 in order to better depict the details of screened tray 40 and cover tray 20. Referring to FIG. 4, screened tray 40 may have profiles to assist with stacking, such as stops 60 and indents 52 on opposite edges 58 and 54, respectively. Stops 60 and indents 52 may be spaced and sized to engage overlying or underlying trays 20 and/or 40. Other profile shapes may be used. As shown, referring to FIGS. 5 and 7, stops 60 may be spaced to engage an overlying or underlying tray 20 or 40 when stacked transversely to help stabilize the tray 20 and/or 40 and may cooperate with indents 34 or 52 on cover tray 20 or screened tray 40 peripheral sidewall 24 along edge 36, and supports screened panel 42 at opening 28 of cover tray 20.

Referring to FIG. 4, containment flange 44 surrounds and extends outward from a first face 42a of screened panel 42. Referring to FIG. 1, containment flange 44 is sized to engage opening 28 of cover tray such that screened panel 42 covers and permits airflow through the opening of cavity 26. As depicted, containment flange 44 may be sized and shaped to nest within opening 28 such that containment flange 44 is immediately adjacent to an inner surface 30 of sidewall 24, such as in sufficiently close proximity to prevent any bees that may be present from passing between containment flange 44 and inner surface 30. The configuration of containment flange 44, opening 28, and sidewall 24 may prevent relative lateral movement of screened tray 40 and cover tray 20 when the two are engaged together.

Referring to FIG. 4, support structure 46 extends outward from a second face 42b of screened panel 42, in a direction opposite of containment flange 44. Support structure 46 may be a depending flange, particularly if screened tray 20 is formed as a molded plastic body. Support structure 46 has a plurality of apertures 56 that permit airflow through support structure 46. Referring to FIG. 6, support structure 46 is sized to engage the closed end 22 of cover tray 20 such that cover tray 20 is spaced from screened panel 42, which allows airflow through apertures 56 to reach screened panel 42 of screened tray 40. Closed end 22 may nest within support structure 46 such that a peripheral shoulder 32 of closed end 22 engages a remote edge 58 of support structure 46. As depicted, support structure 46 may surround screened panel 42.

Referring to FIG. 1, cover tray 20 and screened tray 40 may be rectangular in shape, where both have a length and width that are substantially similar, such that they are engageable to enclose cavity 26 to retain bees within, and to be stacked either in alignment with, or perpendicular to, adjacent sets of trays 10.

Referring to FIG. 5, remote edge 58 of support structure 46 my have stops 60 that cooperate with indents 34 of closed end 22 to support cover tray 20 perpendicularly relative to screened tray. As shown, indents 34 are sized to receive a portion of remote edge 58 of support structure 46 to prevent lateral movement in a first direction, and stops 60 are configured to engage peripheral sidewall 24 of cover tray to prevent movement in a second direction perpendicular to the first direction. It will be understood that indents 34 and stops 60 may have different shapes that similarly prevent such lateral movement.

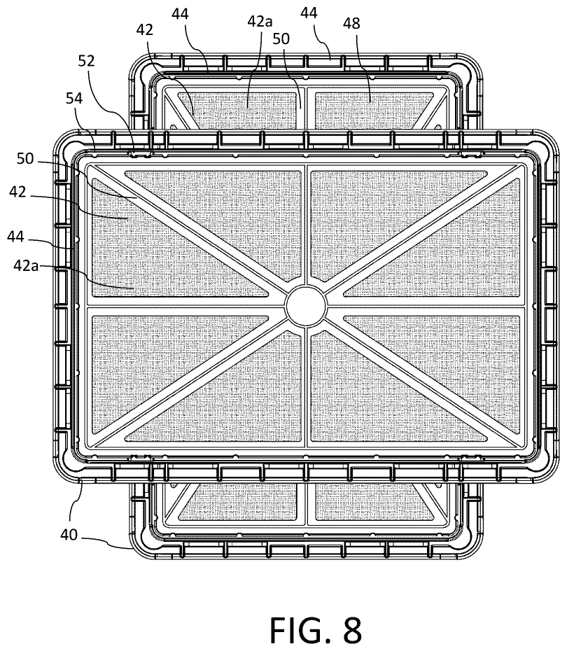

Screened tray 40 may be invertible, such that screened tray 40 may be used to enclose cavity 26 as shown in FIG. 1, or, referring to FIGS. 7 and 8, oriented with containment flange 44 extending up to define a containment tray cavity 41 as shown in FIGS. 7 and 8. Multiple screened trays 40 may be stacked in a perpendicular arrangement, which increases airflow to containment tray cavities 41. This may be useful if containment tray cavities 41 are used to help incubate bees at an early stage. Referring to FIG. 7, indents 52 along distal edge 54 may be sized to receive a portion of remote edge 58 of support structure to prevent lateral movement in a first direction, and stops 60 may be configured to engage distal edge 54 of containment flange 44 to prevent lateral movement in a second direction.

Referring to FIGS. 7 and 8, in a first stage, bee larva (not shown) may be placed in containment tray cavities 41, and multiple screened trays 40 may be stacked perpendicularly. In this arrangement, airflow is able to reach the larva in containment tray cavities 41 through screened panels 42 and apertures 56 in support structures 46, which may be useful for temperature and humidity control, fumigation to control pests, etc.

Referring to FIG. 5, once the larva have reached a certain stage and it is necessary to enclose them, cover trays 20 may be placed over screened trays 40 as a set of trays 10, and sets of trays 10 are inverted such that, for each sets of trays, screened tray 40 is a lid for cover tray 20, and stacked transversely to continue to allow airflow through screened trays 40 via screened panels 42 and apertures 56, with adjacent sets of trays 10 spaced apart by support structures 46 to allow for more airflow. While set of trays 10 may be inverted at any time, inverting set of trays 10 when cover trays 20 are installed may reduce reduces the number of disruptions to the bee larva, and also allows heat generated by the larva to escape through a screen above the larva, rather than be trapped beneath a closed surface as would be the case if cover trays 20 were placed over screened trays 40 in the stack.

Referring to FIG. 6, once the larva have developed to a stage that they may be deployed in a field, sets of trays 10 may be stacked such that adjacent sets 10 are aligned to reduce the volume of the stacks required for transport. As shown, the height is also reduced as the bottom of cover tray 20 nests within screened tray 40, however airflow to bees is still permitted via apertures 56 to maintain bee health during transport and deployment.

The bees may be deployed by removing screened trays 40 and placing cover tray 20 in a bee shelter (not shown). Preferably, as bees tend to fly toward light, cover tray 20 is opaque and dark colored to help encourage bees to fly up and out of cavity 26.

Trays 20 and 40 are preferably made from a sufficiently strong material to support the weight of the stack, This allows trays 20 and 40 to be stacked during use and storage without the need for shelving or racks for storage or transport. This reduces the amount of space required and provides more flexibility for storage and transport. In addition, either screened trays 40 or cover trays 20 may be stacked separately, such as when only screened trays 40 are used during the first stage of incubation, or when only cover trays 20 are used to deploy bees in shelters.

In this patent document, the word “comprising” is used in its non-limiting sense to mean that items following the word are included, but items not specifically mentioned are not excluded. A reference to an element by the indefinite article “a” does not exclude the possibility that more than one of the elements is present, unless the context clearly requires that there be one and only one of the elements.

The scope of the following claims should not be limited by the preferred embodiments set forth in the examples above and in the drawings, but should be given the broadest interpretation consistent with the description as a whole.

What is claimed is:

1. A set of trays, comprising:
a cover tray having a closed end surrounded by a peripheral sidewall that defines a cavity, the cavity having an opening opposite the closed end;
a screened tray comprising:
a screened panel that permits airflow therethrough;
a containment flange that surrounds and extends outward from a first face of the screened panel, the containment flange being sized to engage the opening of the cover tray such that the screened panel covers and permits airflow through the opening of the cavity; and
a support structure that extends outward from a second face of the screened panel opposite the containment flange, the support structure having apertures that permit airflow through the support structure, the support structure being sized to engage the closed end of the cover tray at a position spaced from the screened panel such that the apertures are disposed between the closed end of the cover tray and the screened panel.

2. The set of trays of claim 1, wherein the containment flange is sized to nest within the opening of the cavity and immediately adjacent to an inner surface of the peripheral sidewall.

3. The set of trays of claim 1, wherein:
the support structure surrounds the screened panel and comprises a remote edge; and
the closed end of the cover tray is sized to nest within the support structure and comprises a peripheral shoulder that is sized to engage the remote edge of the support structure.

4. The set of trays of claim 3, wherein:
each of the cover tray and the screened tray are rectangular; and
the closed end further comprises indents and the remote edge of the support structure comprises stops, the indents and stops cooperating to support the cover tray perpendicularly relative to the screened tray.

5. The set of trays of claim 4, wherein the containment flange of the screened tray comprises indents that cooperate with the stops of the support structure to support a plurality of screened trays stacked transversely.

6. The set of trays of claim 1, wherein the screened tray comprises a stepped profile that engages the peripheral sidewall of the cover tray and supports the screened panel of the screened tray at the opening of the cavity of the cover tray.

7. The set of trays of claim 1, wherein the screened tray is invertible, such that the containment flange and the screened panel define a containment tray cavity.

8. A stack of trays, comprising at least an underlying set of trays and an overlying set of trays, each of the underlying set of trays and the overlying set of trays comprising a cover tray and a screened tray, wherein:
each cover tray comprises a length, a width, and a closed end surrounded by a peripheral sidewall that defines a cavity having an opening opposite the closed end; and
each screened tray comprises a length and a width substantially the same as the length and the width of the cover tray, and further comprising:
a screened panel that permits airflow therethrough;
a containment flange that surrounds and extends outward from a first face of the screened panel, the containment flange being received within the opening of the cover tray such that the screened panel covers and permits airflow through the opening of the cavity; and
a support structure that extends outward from a second face of the screened panel opposite the containment flange, the support structure having apertures that permit airflow through the support structure, the support structure being configured to engage the closed end of the cover tray in a first position in which the cover tray is substantially aligned with the screened tray and the cover tray is spaced from the screened panel such that the apertures are disposed between the closed end of the tray and the screened panel, and in a second position in which the cover tray is substantially perpendicular to the screened tray;
wherein the closed end of the cover tray of the overlying set of trays engages the support structure of the screened tray of the underlying set of trays.

9. The stack of trays of claim 8, wherein the containment flange of each screened tray is sized to nest within the opening of the cavity and immediately adjacent to an inner surface of the peripheral sidewall of each cover tray.

10. The stack of trays of claim 8, wherein the support structure comprises a spacer flange that surrounds the screened panel, and the closed end of each cover tray is configured to nest within the spacer flange with a shoulder that is sized engages a remote edge of the spacer flange.

11. The stack of trays of claim 10, wherein the closed end of each cover tray further comprises indents and the remote edge of the support structure of each screened tray comprises stops, the indents and stops cooperating to support the cover tray of the overlying set of trays in the second position on the screened tray of the underlying set of trays.

12. The stack of trays of claim 8, wherein each screened tray comprises a stepped profile that is sized to engage the peripheral sidewall of each cover tray and support the screened panel of each screened tray at the opening of the cavity of each cover tray.

13. The stack of trays of claim 8, wherein each screened tray is invertible, such that the containment flange and the screened panel define a containment tray cavity in a first orientation and encloses the cavity of the cover tray in a second orientation.

14. A method of incubating bees, comprising:
placing bee larva in a plurality of screened trays, each screened tray comprising:
a length;
a width;
a screened panel that permits airflow therethrough;
a containment flange that surrounds and extends outward from a first face of the screened panel; and
a support structure that extends outward from a second face of the screened panel opposite the containment flange, the support structure having apertures that permit airflow through the support structure;
wherein the containment flange comprises a first edge profile that cooperates with a second edge profile of the support structure, the first edge profile and the second edge profile permitting an overlying screened tray to be supported transversely on an underlying screened tray;
stacking the plurality of screened trays in a first stack such that adjacent trays in the stack are positioned in a transverse orientation; and
at a predetermined stage of development of the bee larva, forming a plurality of sets of trays by installing a cover tray on each of the screened trays, each cover tray comprising a length and a width that are substantially the same as the length and the width of the screened tray, and a closed end surrounded by a peripheral sidewall that defines a cavity having an opening opposite the closed end, wherein the containment flange of the screened tray is received within the cavity of the cover tray.

15. The method of claim 14, wherein the plurality of sets of trays are stacked in a second stack in which the support structure and the closed ends of adjacent sets of trays are engaged and such that adjacent sets of trays are positioned in a transverse orientation, wherein the closed end of each cover tray has a third edge profile that cooperates with the second edge profile of the support structure in the transverse orientation.

16. The method of claim 15, wherein the plurality of sets of trays in the second stack are oriented such that, for each set of trays, the screened tray is a lid for the cover tray.

17. The method of claim 15, further comprising the steps of:
at a further stage of development, placing the plurality of sets of trays in a third stack in which the support structures and the closed ends of adjacent sets of trays are engaged such that adjacent sets of trays are aligned;
transporting the third stack to an area to be pollinated; and
for each set of trays and with the cover tray positioned below the screened tray such that developed bee larva are in the cover tray, removing the screened tray and placing the cover tray at a desired location in the area to be pollinated.

18. The method of claim 15, wherein, when the plurality of sets of trays are placed in the third stack, the closed ends of the cover trays nest within the support structures of an adjacent set of trays, the closed ends comprising a peripheral shoulder that engages a remote end of the support structure.

19. The method of claim 12, wherein the containment flange of each screened tray is sized to nest within the opening of the cavity of the cover tray and immediately adjacent to an inner surface of the peripheral sidewall.

20. The method of claim 12, wherein, in the first stack, the containment flange and the screened panel define a containment tray cavity of each screened tray.
